(19) United States
(12) Patent Application Publication
(10) Pub. No.: US 2016/0038549 A1
(43) Pub. Date: Feb. 11, 2016

(54) METHOD FOR PROMOTING INSULIN SECRETION BY USING COMPOUNDS AND EXTRACTS ISOLATED FROM ANTRODIA CAMPHORATA

(71) Applicant: TAI LIN TSENG, Taipei City (TW)

(72) Inventors: MAO TIEN KUO, Taipei City (TW); WAN PING TSENG, Taipei City (TW); TAI LIN TSENG, Taipei City (TW); YIN YU KUO, Taipei City (TW)

(21) Appl. No.: 14/821,121

(22) Filed: Aug. 7, 2015

(30) Foreign Application Priority Data
Aug. 11, 2014 (TW) 103127486
Nov. 21, 2014 (TW) 103140521

(51) Int. Cl. A61K 36/07; A61K 31/215; A61K 31/122

(52) U.S. Cl. CPC A61K 36/07; A61K 31/122; A61K 31/215

(57) ABSTRACT

The present invention discloses a method for promoting insulin secretion by using compounds and extracts isolated from Antrodia camphorata in manufacturing medicaments for promoting insulin secretion, wherein the compounds is represented by formula (I):

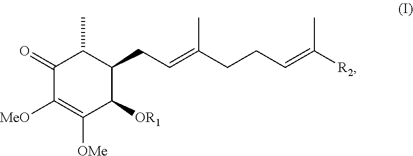

wherein R₁ is a hydrogen atom or acetyl group, R₂ is

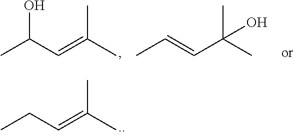

### CROSS-REFERENCE TO RELATED APPLICATION

This application claims the priority of Taiwanese patent application No. 103127486, filed on Aug. 11, 2014 and Taiwanese patent application No. 103140521, filed on Nov. 21, 2014, which are incorporated herewith by reference.

### BACKGROUND OF THE INVENTION

1. Field of the Invention

The present invention relates to a medical use of compounds and extracts isolated from Antrodia camphorata, especially a method for promoting insulin secretion by using the compounds and extracts isolated from Antrodia camphorata.

2. The Prior Arts

Antrodia camphorata is also called Chang-Zhi, Niu Chang-Gu, red camphor mushroom and the like, which is a perennial mushroom belonging to the order Aphyllophorales, the family Polyporaceae. Antrodia camphorata is a unique Formosan fungal growing on the inner wall of the rotten cavity of wood of Cinnamomum kanehirai Hay. Cinnamoum kanehirai Hay is rarely distributed and overcut unlawfully, which makes Antrodia camphorata parasitizing inside the tree in the wild become even rare. The price of Antrodia camphorata is very expensive due to the extremely slow growth rate of the fruiting body that only grows between June to October.

Triterpenoids are the most studied components among the numerous compositions from Antrodia camphorata. Triterpenoids are the summary terms for natural compounds, which contain 30 carbon atoms with the pent acyclic or hex acyclic structures. The bitter taste of Antrodia camphorata is from the components of triterpenoids. Three novel ergostane-type triterpenoids (antcin A, antcin B, antcin C) were isolated by Cherng et al. from the fruiting bodies of Antrodia camphorata (Cherng, I. H., and Chiang, H. C. 1995. Three new triterpenoids from Antrodia cinnamomea. J. Nat. Prod. 58:365-371). Three new compounds zhankuic acid A, zhankuic acid B and zhankuic acid were extracted from the fruiting bodies of Antrodia camphorata with ethanol by Chen et al. (Chen, C. H., and Yang, S. W. 1995. New steroid acids from Antrodia cinnamomea, —a fungus parasitic on Cinnamomum micranthum. J. Nat. Prod. 58:1655-1661). In addition, Cherng et al. also found three other new triterpenoids from the fruiting bodies of Antrodia camphorata, which are sesquiterpene lactone and 2 biphenyl derived compounds, 4,7-dimethoxy-5-methy-1,3-benzodioxole and 2,2′,5,5′-teramethoxy-3,4,3′,4′-bi-methylenedioxy-6,6′-dimethylbiphenyl (Chiang, H. C., Wu, D. P., Cherng, I. W., and Ueng, C. H. 1995. A sesquiterpene lactone, phenyl and biphenyl compounds from Antrodia cinnamomea. Phytochemistry. 39:613-616). In 1996, four novel ergostane-type triterpenoids (antcins E and F and methyl antcinates G and H) were isolated by Cherng et al. with the same analytic methods (Cherng, I. H., Wu, D. P., and Chiang, H. C. 1996. Triteroenoids from Antrodia cinnamomea. Phytochemistry. 41:263-267). And two kinds of ergostane related steroids, zhankuic acids D and E together with three lanosta related triterpenes, 15 alpha-acetyl-dehydrosulphurenic acid, dehydroeburicoic acid, dehydrosulphurenic acid were isolated by Yang et al. (Yang, S. W., Shen, Y. C., and Chen, C. H. 1996. Steroids and triterpenoids of Antrodia cinnamomea—a fungus parasitic on Cinnamomum micranthum. Phytochemistry. 41:1389-1392).

Diabetes mellitus belongs to endocrine metabolic diseases and is a disease with metabolic derangement of sugar, fat, and protein due to absolutely or relatively deficient secretion of insulin. Clinically, this disease is classified into type I and type II. Type I diabetes is insulin-dependent diabetes mellitus (IDDM) and mainly caused by autoimmunity. Because beta-cells in pancreas are injured, the patients' body cannot make insulin and this leads amount of insulin to be absolutely insufficient. Type II diabetes is noninsulin-dependent diabetes mellitus (NIDDM). The insulin cells in the patients' bodies still have the function of secreting insulin. Just because the insulin receptors in target cells (corresponding cells) are resistant or insensitive to insulin, insulin is not released when needed or the combining capability between receptors and insulin is decreasing. Although the amount of insulin is at a normal level, but cannot be satisfied with the requirement for maintaining normal metabolism, and thus sugar is raised in blood and urine. This condition is called insulin relatively deficiency.

Although it is known by many recent experiments that Antrodia camphorata has various medical effects, but has not been proved for specific extracts/compounds having the effect of promoting insulin secretion. Consequently, the applications in the related fields about studying Antrodia camphorata should be further clarified.

### SUMMARY OF THE INVENTION

Accordingly, the inventors have studied quite a lot on the extracts/compounds from Antrodia camphorata and found certain of them having the effect for insulin secretion.

An objective of the present invention is to provide a method for promoting insulin secretion by using compounds isolated from Antrodia camphorata, comprising administering to a subject in need thereof an effective amount of compounds isolated from Antrodia camphorata, wherein the compound is represented by formula (I):

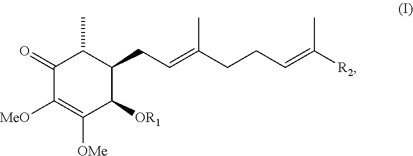

wherein R₁ is a hydrogen atom or acetyl group, and R₂ is

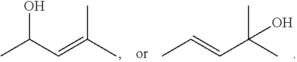

Preferably, wherein R₁ in the compound is a hydrogen atom, R₂ is

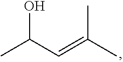

and the compound is represented by formula (II):

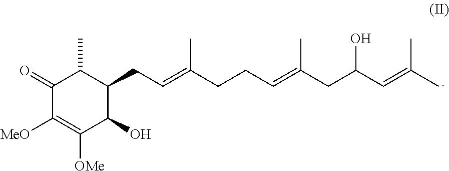 [SMILES: COC1=C(OC)[C@H](O)[C@H](C/C=C(\C)CC/C=C(\C)CC(O)C=C(C)C)[C@@H](C)C1=O]

Preferably, wherein R₁ in the compound is an acetyl group, R₂ is

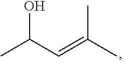

and the compound is represented by formula (III):

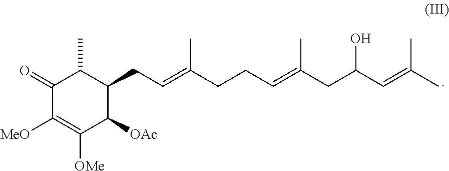 [SMILES: COC1=C(OC)[C@H](OC(C)=O)[C@H](C/C=C(\C)CC/C=C(\C)CC(O)C=C(C)C)[C@@H](C)C1=O]

Preferably, wherein R₁ in the compound is a hydrogen atom, R₂ is

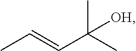

and the compound is represented by formula (IV):

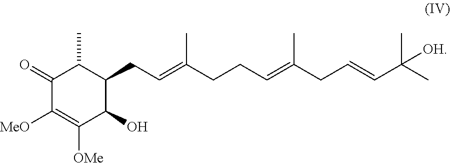 [SMILES: COC1=C(OC)[C@H](O)[C@H](C/C=C(\C)CC/C=C(\C)C/C=C/C(C)(C)O)[C@@H](C)C1=O]

Preferably, wherein R₁ is a hydrogen atom, R₂ is

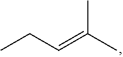

and the compound is represented by formula (V):

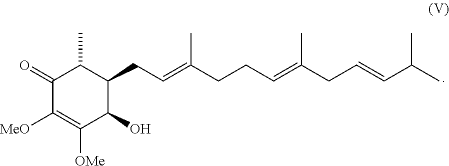 [SMILES: COC1=C(OC)[C@H](O)[C@H](C/C=C(\C)CC/C=C(\C)C/C=C/C(C)C)[C@@H](C)C1=O]

The present invention further provides a method for promoting insulin secretion by using extracts isolated from Antrodia camphorata.

Preferably, wherein the extracts are extracted and then obtained by the following steps: the mycelium, fruiting body, or mixture of both taken from Antrodia camphorata are extracted twice by ten-fold ethanol and then the ethanol solutions are merged to concentrate to a crude product; and then the crude product is operated triple by partition extract with dichloromethane/water (1:1) to form a dichloromethane layer and a water layer, wherein the dichloromethane layer and the water layer are the extracts from Antrodia camphorata.

### BRIEF DESCRIPTION OF THE DRAWINGS

FIGS. 1A-1B were represented by MIN6 cells viability assay for the extracts from Antrodia camphorata ANCA-D and ANCA-W, respectively.

FIGS. 2A-2C were represented by MIN6 cells viability assay for Antrocamol LT1, Antrocamol LT2, and Antrocamol LT3, respectively.

FIGS. 3A-3C were represented by the glucose-stimulated insulin secretion (GSIS) assay for the extracts from Antrodia camphorata: ANCA-D and ANCA-W, and the compounds from Antrodia camphorata: Antrocamol LT1, Antrocamol LT2, Antrocamol LT3, and Antroquinonol respectively.

### DETAILED DESCRIPTION OF THE PREFERRED EMBODIMENT

Extraction of Components from Antrodia camphorata

The mycelium, fruiting body or mixture of both taken from Antrodia camphorata (1.0 kg) were extracted ethanol (10 L×2) and then the ethanol solutions were merged to concentrate to a crude product (LT-E, about 230 g). The crude product was operated triple by partition extraction with dichloromethane/water (1:1) to form a dichloromethane layer (ANCA-D, about 102.6 g) and a water layer (ANCA-W, about 127.4 g). Then, dichloromethane layer (6.0 g) was separated to four layers (ANCA-E-D-1, ANCA-E-D-2, ANCA-E-D-3, and ANCA-E-D-4) by silica column chromatography with eluents (n-hexane/dichloromethane (1:4), dichloromethane, and methanol/dichloromethane (5:95)). Each of the aforementioned four layers was obtained by the following: the combination of the hexane/dichloromethane (1:4) and the dichloromethane eluates (ANCA-E-D-1); first part of the methanol/dichloromethane (5:95) eluate (ANCA-E-D-2); second part of the methanol/dichloromethane (5:95) eluate (ANCA-E-D-2); the methanol eluate in the end (ANCA-E-D-4).

Compounds from Antrodia camphorata: Antrocamol LT1, Antrocamol LT2, and Antrocamol LT3

The compounds from Antrodia camphorata: Antrocamol LT1, Antrocamol LT2, and Antrocamol LT3 were three new compounds purified from Antrodia camphorata by the inventors. However, the purification and structural identification of the aforementioned compounds had been disclosed in the previous applications. As a result, these compounds were not discussed again, and only the formula and the brief data of these compounds were provided by the following contexts:

Antrocamol LT1 is a colorless liquid product, and it was analyzed and found that its molecular formula was C₂₄H₃₈O₅ with a molecular weight of 406. The complete name of this product was called 4-hydroxy-5-[9-hydroxy-3,7,11-trimethyldodeca-2,6,10-trienyl]-2,3-dimethoxy-6-methyl-cyclohex-2-enone. Its structure was represented by formula (II):

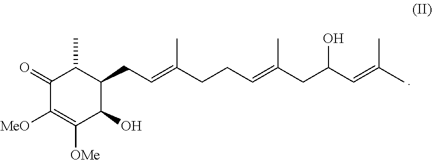 [SMILES: COC1=C(OC)[C@H](O)[C@H](C/C=C(\C)CC/C=C(\C)CC(O)C=C(C)C)[C@@H](C)C1=O]

Antrocamol LT2 is a colorless liquid product, and it was analyzed and found that its molecular formula was C₂₆H₄₀O₆ with a molecular weight of 448. The complete name for this product was called 4-acetoxy-5-[9-hydroxy-3,7,11-trimethyldodeca-2,6,10-trienyl]-2,3-dimethoxy-6-methyl-cyclohex-2-enone. Its structure was represented by formula (III):

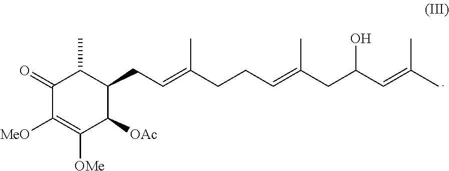 [SMILES: COC1=C(OC)[C@H](OC(C)=O)[C@H](C/C=C(\C)CC/C=C(\C)CC(O)C=C(C)C)[C@@H](C)C1=O]

Antrocamol LT3 is a colorless liquid product, and it was analyzed and found that its molecular formula was C₂₄H₃₈O₅ with a molecular weight of 448. The complete name for this product was called (4R,5R,6R)-4-hydroxy-5-[(2E,6E,9E)-11-hydroxy-3,7,11-trimethyldodeca-2,6,9-trienyl]-2,3-dimethoxy-6-methylcyclohex-2-enone. Its structure was represented by formula (IV):

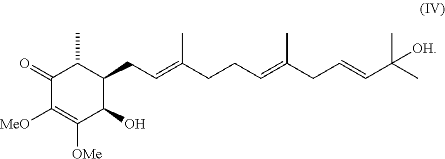 [SMILES: COC1=C(OC)[C@H](O)[C@H](C/C=C(\C)CC/C=C(\C)C/C=C/C(C)(C)O)[C@@H](C)C1=O]

Antroquinonol was called (4R,5R,6R)-4-hydroxy-5-[(2E,6E,9E)-11-hydroxy-3,7,11-trimethyldodeca-2,6,9-trienyl]-2,3-dimethoxy-6-methylcyclohex-2-enone. Its structure was represented by formula (V):

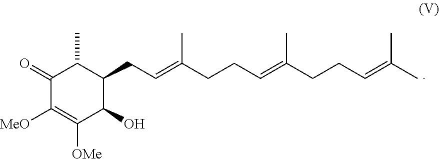 [SMILES: COC1=C(OC)[C@H](O)[C@H](C/C=C(\C)CC/C=C(\C)CCC=C(C)C)[C@@H](C)C1=O]

The aforementioned Antrocamol LT1, Antrocamol LT2, Antrocamol LT3, and Antroquinonol had a similar primary structure, and their general formula was represented by formula (I):

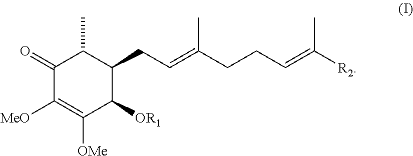

Wherein R₁ is a hydrogen atom or an acetyl group, R₂ is

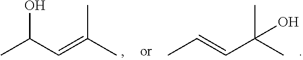

Wherein R₁ in the compound is a hydrogen atom, R₂ is

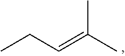

and the compound is represented by formula (V):

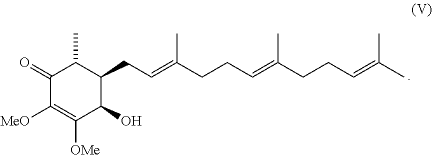 [SMILES: COC1=C(OC)[C@H](O)[C@H](C/C=C(\C)CC/C=C(\C)CCC=C(C)C)[C@@H](C)C1=O]

For detailed information about extraction and purification of four kinds of the aforementioned compounds from Antrodia camphorata, they can be referred to the inventor's previous relative applications. Since it has been disclosed that said compounds contain various medical effects in the previous applications. In the present invention, relative experiments with the four compounds are proceeded in order to determine their influences for insulin secretion, and to study that whether those compounds have medical potentials for relative insulin secretion diseases such as diabetes mellitus.

MTT Cell Viability Assay with Extracts/Compounds from Antrodia camphorata

MIN6 mouse beta insulinoma cell line was analyzed by MTT cell viability assay respectively. The MIN6 mouse insulinoma beta cell line is an insulinoma cell line which comes from sarcoma virus 40 transfecting non-obese diabetic mice (NOD mice) by T cells, and is a mouse insulinoma cell line having physiological characteristics of insulinoma beta cell.

### MIN6 Cell Culture

MIN6 cell line was cultured in a high glucose DMEM with 15% fetal bovine serum, 50 μmol/L mercaptoethanol, 100 U/ml penicillin, and 100 mg/ml streptomycin. The medium was replaced the next day.

### MTT Assay

MIN6 cells were prepared for 1×10⁵/ml cell liquid suspensions, and then the suspensions were incubated in a 96-well plate with 100 μl in each well. After cell attachment, the medium was replaced with a 0.2% serum medium and then the cells were cultured for 24 hours in order to keep the cells at rest. Then, 200 mg/L BSA medium and the extracts/compounds from Antrodia camphorata with a variety of predetermined concentrations (experimental groups) were added to react for 24 hours, respectively, and then thapsigargin was added to react for 24 hours. A control group was prepared by adding 100 μl medium and then incubating in a constant temperature incubator at 37° C., 5% CO₂ for a period of time. 4 hours before the end of the incubating process, 20 μl MTT (0.5 mg/ml) was added into each well, the samples continued being incubated for 4 hours, and then crystalline dissolved fully with vibration. The absorbance (A) at wavelength of 570 nm was determined respectively by a reader. Then the cell viability was calculated by the following formula: the cell viability=(the average of A570 in the experimental group/the average of A570 in the control group)×100%.

Effects of Extracts from Antrodia camphorata, ANCA-D and ANCA-W for MIN6 Cell Viability

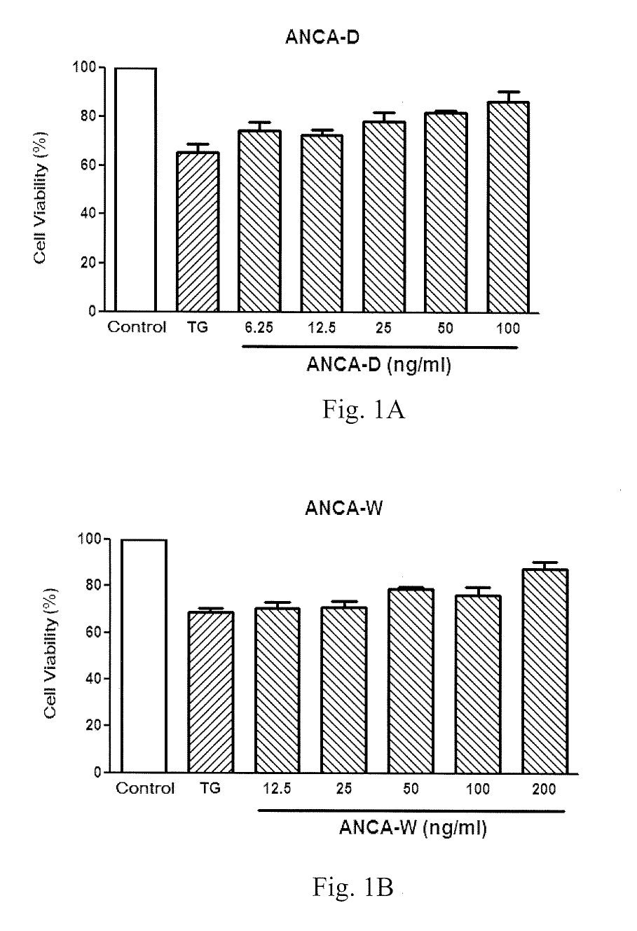

Referring to FIG. 1A, the extraction from ANCA-D in different concentrations (6.25, 12.5, 25, 50, 100 ng/ml) was added into cells in each group respectively to react for 24 hours. Then, thapsigargin (TG) was added to react for 24 hours and the cell viability of each group was determined after the reaction. Thapsigargin is an endoplasmic reticulum calcium pump inhibitor which can promote depletion of calcium ions from the endoplasmic reticulum and then open store-operated channels to lead the calcium ions to increase in cells. Then, insulin secretion could be inhibited.

The results were represented by FIG. 1A, the cells in each group were pretreated with the extraction from Antrodia camphorata in the different concentrations for 24 hours, and then TG was added to react for 24 hours. It exhibited that compared with the control group in which only TG was added, the cell viability had an increasing tendency respectively in the experimental groups pretreated with higher concentrations of the extraction from Antrodia camphorata ANCA-D. By the result, it indicated that the extractions from Antrodia camphorata ANCA-D and ANCA-W could slightly increase MIN6 cell viability, and perform slightly a positive correlation with the concentrations.

Effects of Antrodia camphorata Extracts Antrocamol LT1, Antrocamol LT2 and Antrocamol LT3 for MIN6 Cell Viability

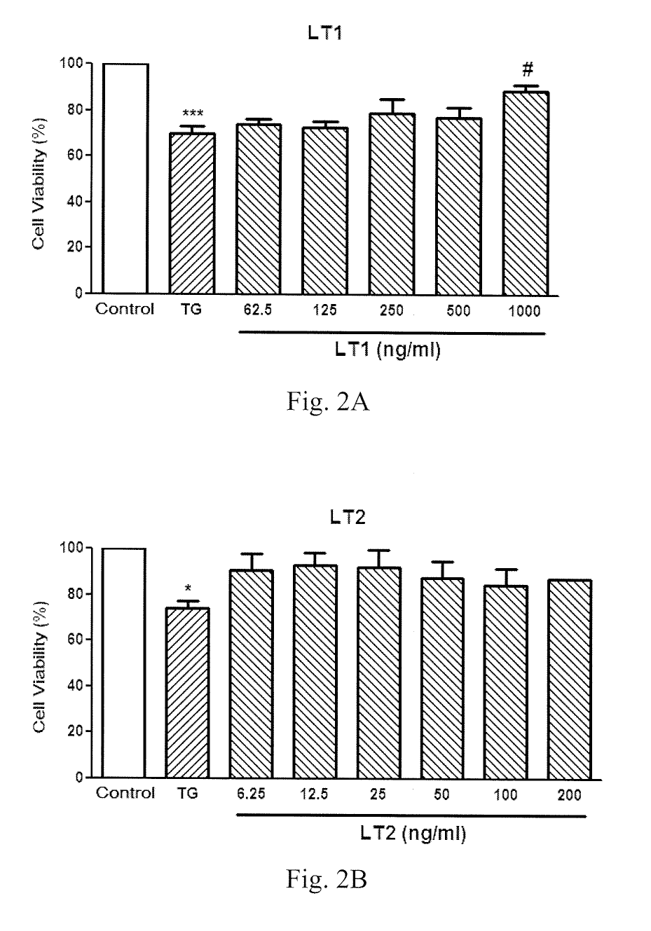

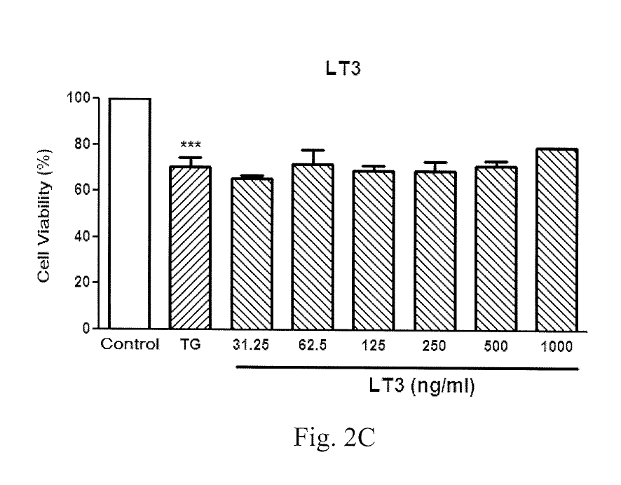

Referring to FIGS. 2A-2C, the figures indicated respectively the effects of the extract from Antrodia camphorata Antrocamol LT1, Antrocamol LT2 and Antrocamol LT3 for the MIN6 cell viability. As represented by FIG. 2A, compared with the control group in which only TG was added, Antrocamol LT1 could increase the MIN6 cell viability and the increasing tendency was more significant especially in the concentrations of 250, 500, and 1000 ng/ml.

Referring to FIG. 2B, compared with the control group in which only TG was added, Antrocamol LT2 could significantly increase the MIN6 cell viability in lower concentrations, especially in 6.25˜25 ng/ml. However, in the group of concentration higher than 50 ng/ml, the MIN6 cell viability began to slightly decrease.

Referring to FIG. 2C, compared with the group in which only TG was added, in the group of Antrocamol LT3 concentrations lower than 500 ng/ml, it seemed that the concentrations did not significantly influence the MIN6 cell viability, and the viability began increasing in the concentration of 1000 ng/ml.

### Glucose-Stimulated Insulin Secretion (GSIS) Assay

In order to examine the influences of the aforementioned extracts and compounds from Antrodia camphorata for insulin secretion, GSIS assay was performed with MIN6 cell. First, MIN6 cell was incubated in 1×10⁵/ml concentration in a 24-well plate and a 15% FBS DMEM medium at 37° C., 5% CO₂. After cell attachment, 200 mg/1 BSA medium and the extracts and compounds from Antrodia camphorata in different concentrations were added into each well to react for 24 hours. 5.5 mM/16.7 mM glucose was further added as stimulation and then insulin was determined by ELISA. In the same time, total cellular protein in each well was extracted for a calibration by determining protein concentration with BCA assay.

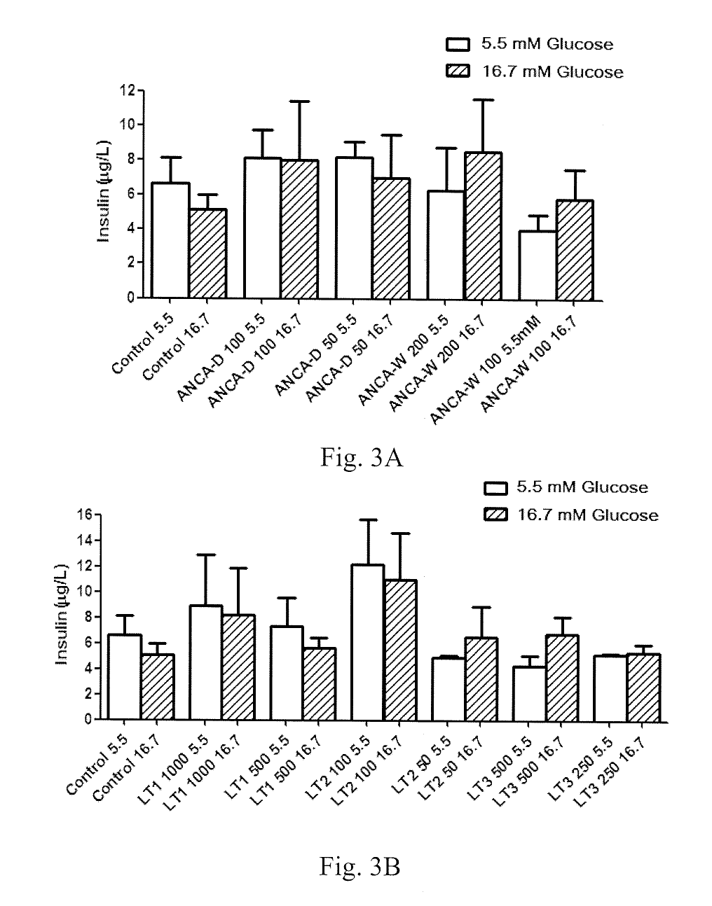

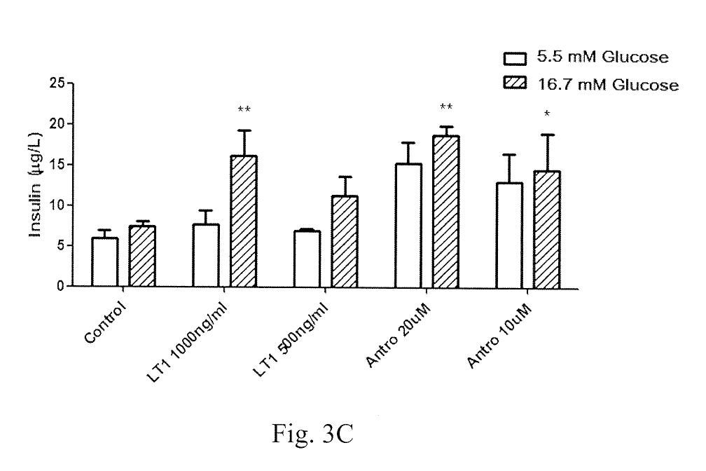

Referring to FIG. 3A, it indicated the results of insulin secretion which was pretreated with different concentrations of the extracts from Antrodia camphorata: ANCA-D (100 ng/ml and 50 ng/ml) and ANCA-W (200 ng/ml and 100 ng/ml), and then was stimulated with 5.5 mg/16.7 mg glucoses. As represented by the figure, compared with the control group, in the experimental groups pretreated with 100 ng/ml and 50 ng/ml ANCA-D, the results of insulin secretion increased were observed in both high (16.7 mM) and low (5.5 mM) concentrations of glucose. However, compared with the control group, the insulin secretion did not increase in the groups pretreated with 200 ng/ml and 100 ng/ml ANCA-W. According to the experimental results, the same assay was further performed with the following three compounds: Antrocamol LT1, Antrocamol LT2, and Antrocamol LT3 purified from ANCA-D.

Referring to FIG. 3B, it indicated the results of insulin secretion which was pretreated with different concentrations of compounds from Antrodia camphorata: Antrocamol LT1 (1000 ng/ml and 500 ng/ml), Antrocamol LT2 (100 ng/ml and 50 ng/ml), and Antrocamol LT3 (500 ng/ml and 250 ng/ml), and then was stimulated with 5.5 mM/16.7 mM glucose. As represented by the figure, the group of Antrocamol LT2 (100 ng/ml) indicated that the effect of promoting insulin secretion was the most significant and was about 2 times higher than the control group. Although the effect of Antrocamol LT1 (1000 ng/ml and 500 ng/ml) was not significant than Antrocamol LT2 in promoting insulin secretion, but a slightly increasing tendency was still observed. In the group of Antrocamol LT3, there was also a slightly tendency for promoting insulin secretion by pretreating with 250 ng/ml. According to the aforementioned results, it could be seen that the conditions of the three compounds promoting insulin secretion were different from each other. For example, the effect of Antrocamol LT2 was quiet significant at 100 nM/ml concentration but the effect was not observed at 50 ng/ml concentration. Alternatively, the effect of Antrocamol LT3 for promoting insulin secretion was observed in high glucose but not low glucose. The differences might be caused by each ingredient involving dissimilar mechanisms, but was not discussed here. However, in objective view, the aforementioned three compounds exhibiting the effects of promoting insulin secretion was still observed in different conditions. Therefore, by further research in the future, the medical compositions with different medical mechanisms will be further developed according to their properties, and they are considered as a medical application for diabetes mellitus and insulin secretion related diseases.

In addition, the inventors further performed the experiments of insulin secretion with Antrocamol LT1 and Antroquinonol which is similar to Antrocamol LT series in structure for insulin secretion.

Referring to FIG. 3C, it indicated the results of insulin secretion which was pretreated with Antrocamol LT1 (1000 ng/ml and 500 ng/ml) and Antroquinonol (abbreviation as Antro) (500 ng/ml and 250 ng/ml) and then was stimulated with 5.5 mM/16.7 mM glucose. As represented by the figure, in the high glucose (16.7 mM)/low glucose (5.5 mM), Antrocamol LT1 (500 ng/ml) exhibited the effect for promoting insulin secretion, and the stimulated effect was more obvious with higher glucose concentration in 16.7 mM for about 2 times higher than the control group. The stimulation was also slightly increased with lower glucose concentration in 5.5 mM. In the group with Antrocamol LT1 (1000 ng/ml), the stimulated effect was more obvious with higher glucose concentration in 16.7 mM, and had a respectively increasing tendency in lower glucose concentration with 5.5 mM. According to the results, it seemed that the effect of Antrocamol LT1 for insulin secretion was efficient in the higher glucose concentration with 16.7 mM.

Furthermore, as the represented figure, it seemed that Antroquinonol was more efficient for stimulating insulin secretion. The groups which were pretreated with Antroquinonol in 10 μm or 20 μm concentrations both had obvious effect for insulin secretion in the higher (16.7 mM)/lower (5.5 mM) glucose concentration (compared with the control group). The group only pretreated with Antroquinonol in 10 μm concentrations could achieve 2 times the amount of insulin secretion, and the group pretreated with Antroquinonol in 20 μm could achieve even 3 times the amount of insulin secretion. By the further experimental results, it was also proved that Antrocamol LT1 and Antroquinonol have well effect for stimulating insulin secretion. However, the relative mechanism should be more studied.

The present invention offers an application of compounds and extracts isolated from Antrodia camphorata in preparation medicaments for treating kidney diseases that actually contains useful value on industry. It is not to be limited in scope by the specific embodiments described herein. Indeed, various modifications of the invention in addition to those described herein will be apparent to those skilled in the art from the foregoing description. Such modifications are intended to fall within the scope of the appended claims.

What is claimed is:

1. A method for promoting insulin secretion by using compounds isolated from Antrodia camphorata, comprising administering to a subject in need thereof an effective amount of compounds isolated from Antrodia camphorata, wherein the compounds are represented by formula (I): 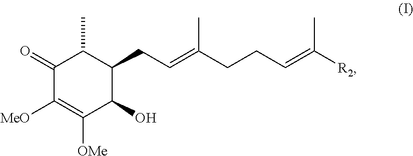
wherein R₁ is a hydrogen atom or acetyl group, R₂ is

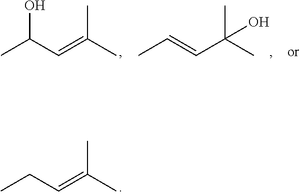

2. The method of claim 1, wherein R₁ in the compound is a hydrogen atom, R₂ is 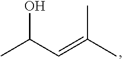
and the compound is represented by formula (II):

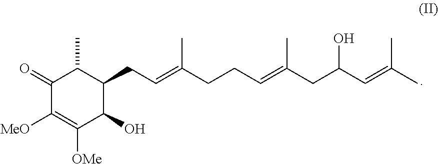 [SMILES: COC1=C(OC)[C@H](O)[C@H](C/C=C(\C)CC/C=C(\C)CC(O)C=C(C)C)[C@@H](C)C1=O]

3. The method of claim 1, wherein R₁ in the compound is an acetyl group, R₂ is 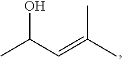
and the compound is represented by formula (III):

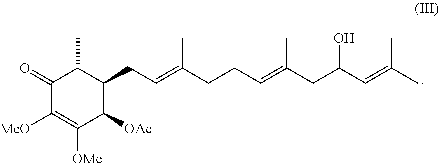 [SMILES: COC1=C(OC)[C@H](OC(C)=O)[C@H](C/C=C(\C)CC/C=C(\C)CC(O)C=C(C)C)[C@@H](C)C1=O]

4. The method of claim 1, wherein R₁ in the compound is a hydrogen atom, R₂ is 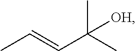
and the compound is represented by formula (IV):

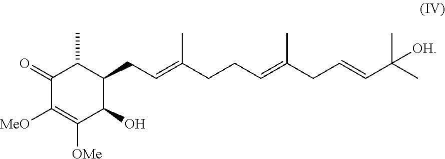 [SMILES: COC1=C(OC)[C@H](O)[C@H](C/C=C(\C)CC/C=C(\C)C/C=C/C(C)(C)O)[C@@H](C)C1=O]

5. The method of claim 1, wherein R₁ is a hydrogen atom in the compound, R₂ is 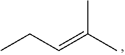
and the compound is represented by (V):

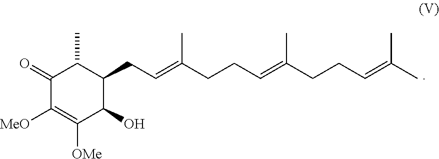 [SMILES: COC1=C(OC)[C@H](O)[C@H](C/C=C(\C)CC/C=C(\C)CCC=C(C)C)[C@@H](C)C1=O]

6. A method for promoting insulin secretion by using extracts from Antrodia camphorata, comprising administering to a subject in need thereof an effective amount of extracts isolated from Antrodia camphorata, wherein the extracts are extracted and then obtained by following steps: the mycelium, fruiting body or mixture of both taken from Antrodia camphorata are extracted twice by ten-fold ethanol; and then the ethanol solutions were merged to concentrate to a crude product; and then the crude product is operated triple by partition extraction with dichloromethane/water (1:1) to form a dichloromethane layer and a water layer, wherein the dichloromethane layer and the water layer are the extracts from Antrodia camphorata.
